(19) United States
(12) Patent Application Publication
(10) Pub. No.: US 2025/0362234 A1
(43) Pub. Date: Nov. 27, 2025

(54) Method and device for quantitatively detecting salt deposits on a metal surface by means of laser-induced plasma spectroscopy

(71) Applicant: Fraunhofer-Gesellschaft zur Förderung der angewandten Forschung eingetragener Verein, München (DE)

(72) Inventors: Peter PLAGEMANN, Bremen (DE); Oliver KRANZ, Düsseldorf (DE); Julian HEINE, Bremen (DE); Theresa BEER, Bremen (DE); Uwe SPECHT, Verden (DE)

(21) Appl. No.: 18/872,605

(22) PCT Filed: Jun. 9, 2023
(86) PCT No.: PCT/EP2023/065459

§ 371 Date: Dec. 6, 2024

(30) Foreign Application Priority Data
Jun. 9, 2022 (DE) 10 2022 114 580.8

(51) Int. Cl. G01N 21/71; G01N 33/20

(52) U.S. Cl. CPC G01N 21/718; G01N 33/20; G01N 2201/06113; G01N 2201/127

(57) ABSTRACT

The invention relates to a device for quantitatively detecting salt deposits on a metal surface by means of laser-induced plasma spectroscopy. The invention also relates to a method for uniformly applying salts to a metal surface (13) or for producing a reference metal surface. The invention further relates to the use of laser-induced plasma spectroscopy for quantitatively detecting salt deposits on a metal surface and the use of a corresponding device for carrying out laser-induced plasma spectroscopy for quantitatively detecting salt deposits on a metal surface.

The present invention relates to a method and to an apparatus for quantitative detection of salt deposits on a metal surface by means of laser-induced plasma spectroscopy. The invention further relates to a method of uniformly contacting a metal surface with salts or of producing a reference metal surface. The invention also relates to the use of laser-induced plasma spectroscopy for quantitative detection of salt deposits on a metal surface, and to the use of an apparatus of the invention for performance of a method of the invention.

The invention is defined in the appended claims. Preferred aspects of the present invention, moreover, will be apparent from the description that follows, including the examples.

Where particular configurations are referred to as being preferred for one aspect of the invention, the corresponding observations will in each case also be applicable to the other aspects of the present invention, mutatis mutandis. Preferred individual features of aspects of the invention (as defined in the claims and/or disclosed in the description) are combinable with one another, and are preferably combined with one another unless the opposite is apparent to the person skilled in the art in the individual case from the present text.

Metal surfaces, especially steel surfaces, must also be free of salts as well as other impurities prior to coating, since there can otherwise be bubble formation in the coating to be applied, which is associated with premature failure of the protective function of the coating. Consequently, in practice, coating of a metal surface typically has to be preceded by demonstration that the limit for acceptable salt deposits assigned to the respective coating material to be used on the metal surface to be coated (often a maximum of 20 mg/m² NaCl equivalent) is not exceeded.

The quantitative content of salt deposits on a metal surface to be coated is determined here in practice by random sampling using what is called the Bresle test. In this test, first of all, the (water-soluble) salts present in a defined area of the metal surface to be examined are dissolved by, in accordance with standard DIN EN ISO 8502-6:2020-08, sticking a chamber patch onto the metal surface to be examined, into which a syringe is used to inject a known amount of distilled water and, after waiting for a particular defined extraction time, drawing it back out of the chamber patch. The content of extracted salt in the water sample is then determined by conductivity measurement in accordance with standard DIN EN ISO 8502-9:2020-12 and typically reported in NaCl equivalents.

There are some drawbacks associated with employment of the Bresle test to ascertain the salt content on metal surfaces. For instance, employment of the Bresle test is comparatively time-consuming and complex and is not automatable because of the procedure outlined above. On employment of the Bresle test, it is also the case that the surface to be examined is contaminated with adhesive residues by virtue of the need to stick on a chamber patch, as a result of which, after employment of the Bresle test, it is necessary to clean the local surface again prior to application of a coating on the surface. The Bresle test also does not permit differentiation of individual salt components on the metal surface to be examined. In addition, in the case of employment of the Bresle test, comparatively large areas (of about 3-5 cm²) are covered for each “measurement point”, which is at the expense of the resolution of the overall measurement result when multiple individual measurements are performed as customary (in other words, the Bresle test cannot detect point salt deposits, and any salt deposits will only ever be narrowed down to a particular larger area).

Against this background, it was a primary object of the present invention to provide a simple and efficient method of quantitative detection of salt deposits on a metal surface, which overcomes the abovementioned disadvantages of the methodology known from the prior art.

It was a further object of the present invention to specify an apparatus for simple and rapid quantitative detection of salt deposits on a metal surface, wherein the apparatus should especially be suitable for performance of the method to be provided for quantitative detection of salt deposits on a metal surface.

Further objects will be apparent from the description that follows and the claims.

The primary object of the present invention is achieved by a method of quantitative detection of salt deposits (or of salt-forming chemical elements) on a metal surface by laser-induced plasma spectroscopy (LIPS or LIBS for short), comprising the following steps:
I) focusing a laser beam onto a point on the metal surface to be examined, so as to form a local plasma;
II) spectroscopically analyzing the radiation emitted by the local plasma on cooling, so as to obtain a spectrum for the radiation emitted by the local plasma;
III) identifying (at least) one characteristic spectral line for (at least) one chemical element present in the salt deposit from the spectrum obtained in step II) and determining the area beneath the spectral line;
IV) quantifying the content of salt deposits (or of salt-forming chemical elements) on the metal surface to be examined by comparing the area determined in step III) with areas obtained from calibration measurements,
wherein the calibration measurements are each performed on reference metal surfaces with respectively known contents of salt deposits.

The elemental composition of a surface can be analyzed by laser-induced plasma spectroscopy. This involves focusing a high-energy laser onto the surface to be examined, giving rise to a local plasma that emits radiation on cooling. The emitted radiation can be analyzed spectroscopically, where the wavelengths of the spectral lines discernible in the resultant spectrum permit conclusions as to the element types present on the surface (since the spectral lines of each chemical element each occur at known element-specific wavelengths). The intensity of a spectral line in the form of the size of the area beneath the respective spectral line additionally gives a pointer as to the amount of a chemical element at the surface site examined.

However, it is not possible to infer absolute concentrations (e.g. atom % or ppm) directly from the areas beneath the spectral lines of a spectrum obtained by laser-induced plasma spectroscopy. But as has been shown, quantification of salt deposits or salt-forming chemical elements on a metal surface by laser-induced plasma spectroscopy is possible by comparison or matching of the ascertained areas of one or more characteristic spectral lines of the metal surface examined with reference areas of spectral lines of the same species that have been ascertained on reference metal surfaces having known contents of salt deposits.

In other words, for quantitative detection (i.e. for the determination of absolute concentration values) of salt deposits (or salt-forming chemical elements) on a metal surface by laser-induced plasma spectroscopy, calibration measurements on reference metal surfaces with respectively known contents of salt deposits are required, provided that a linear correlation exists between the salt content or the content of salt-forming chemical elements on a metal surface and the intensity of spectral lines discernible in the LIBS spectrum.

It is precisely this aspect that has to date deterred specialists from considering the methodology of laser-induced plasma spectroscopy for quantitative detection of salt deposits or salt-forming chemical elements on a metal surface, since linear correlation of the intensity of spectral lines of salt-forming chemical elements and the salt content on the metal surface cannot be expected directly, and hence successful employment of laser-induced plasma spectroscopy for quantitative detection of exactly such salt contents on metal surfaces was not predictable.

A further barrier has to date been the provision of reference metal surfaces having defined contents of salt deposits (which are of course required for the calibration measurements to be conducted), since such reference metal surfaces are not directly producible with homogeneous and known concentrations of salt contaminants. For example, according to the current state of knowledge, in the production of such salt-contaminated reference metal surfaces, it was always necessary to assume that controlled salt contamination can simultaneously bring about corrosion of the metal surface and hence can adversely affect or distort the result of the calibration measurement.

However, the inventors have found in the course of their studies that there is indeed a linear correlation between the content of salt deposits or the content of salt-forming chemical elements on metal surfaces and the intensity (the areal extent) of spectral lines from salt-forming chemical elements that are identifiable in LIBS spectra. Furthermore, the inventors have succeeded, by the (mist) spraying of metal surfaces with a salt-containing aerosol (mist) under predefined conditions, in a surprisingly simple and efficient manner, in creating reference metal surfaces having homogeneous and known amounts of salt deposits that are suitable for the calibration measurements of the method of the invention.

By virtue of the considerations and efforts undertaken, the inventors have succeeded in providing, by the method of the invention, a possibility of quantitative detection of salt deposits or of salt-forming chemical elements on metal surfaces which, by comparison with the Bresle test known from the prior art for the present purposes,
is more easily and rapidly performable and is less complex (the determination, if required, can be controlled in a purely instrument-controlled manner and is repeatable in an entirely unproblematic manner at any number of sites on the metal surface to be examined without any great time demands),
leaves behind less surface contamination, if any, on the metal surface to be examined (in the method of the invention, by comparison with the Bresle test, no large-area moistening of the metal surface to be examined is required; there is also no risk with the method of the invention that adhesive residues will remain on completion of measurement),
not only permits a conclusion as to the total salt content on the metal surface to be examined, but simultaneously also enables differentiation of the salt-forming chemical elements present on the metal surface to be examined,
also permits detection and quantitative determination of small concentrations of salt deposits (the accuracies for the determination of salt contents that are obtained by the method of the invention are achievable with the accuracies known from the Bresle test and can even be surpassed),
permits very location-specific analysis of the metal surface to be examined (which, in the case of performance of multiple measurements on the metal surface to be examined, by comparison with the Bresle test, enables better resolution for the overall measurement result).

The expression “metal surface” in the context of the present invention also encompasses metal surfaces having a thin metal oxide layer (caused, for example, by oxidation/corrosion).

Preferably, in step I) of the method of the invention, a pulsed laser beam is focused onto a site on the metal surface to be examined. The use of a pulsed laser beam has the advantage that the plasma formed can be better or more quickly cooled, which is in turn beneficial to the performance of the subsequent step II) of the method of the invention, in which the radiation emitted on cooling of the local plasma is analyzed spectroscopically.

For the determination of the total salt content of a metal surface, it may be sufficient in step III) of the method of the invention to determine the area beneath only a single characteristic spectral line and to use this to infer the total content of salt deposits in step IV). This is the case especially when the type or composition of the salt deposit to be expected is known. If the salt deposit to be expected is, for example, sea salt, it is possible, for example, by determining the sodium content on the surface with reference to a spectral line for sodium or sodium ions which is characteristic for this purpose, to infer the total content of sea salt deposits on the metal surface, since the ratio of the main ions in seawater is always approximately the same and hence the quantification of a single salt-forming chemical element simultaneously permits conclusions as to the content of further salt-forming chemical elements of sea salt.

In other cases, however, it may be advisable to ascertain the quantitative content of salt deposits on a metal surface to be examined with reference to the area contents of two or more characteristic spectral lines, especially when the nature of the salt deposits to be expected is not sufficiently known. The subject matter of the present invention therefore explicitly also includes methods in which salt deposits or salt-forming chemical elements on a metal surface are quantitatively detected by determination of the area beneath more than one characteristic spectral line.

In the context of the present invention, characteristic spectral lines for chemical elements present in the salt deposit generally mean the spectral lines of all salt-forming elements that can be detected on the respective metal surface to be examined.

It is preferably the most intense spectral line of a salt-forming element on the metal surface to be examined in the spectrum which is used for the determination of the content of the particular element. In the context of the invention, however, it is also possible to use any other spectral line of a salt-forming element for quantitative detection, provided that the intensity of the respective spectral line is sufficiently high to enable determination of the area beneath it.

The use of laser-increased plasma spectroscopy offers the advantage here that every measured spectrum can be used to simultaneously indicate or identify the spectral lines of all elements present on the metal surface, and hence the recording of a single spectrum is sufficient to undertake quantitative detection of salt deposits using two or more characteristic spectral lines.

As already elucidated above, the content of salt deposits on the metal surface to be examined is quantified in the course of the method of the invention by comparing the area(s) determined in step III) with areas obtained from calibration measurements for the characteristic spectral lines chosen for the analysis, where the calibration measurements are each conducted on reference metal surfaces with respectively known contents of salt deposits. The (calibration) areas obtained by calibration measurements are likewise ascertained from spectra obtained by laser-induced plasma spectroscopy, preferably using a measurement device of the same type and/or the same measurement conditions for laser-induced plasma spectroscopy for the analysis of the metal surface to be examined and the performance of the calibration measurements.

Moreover, the quantitative content of salt deposits on the reference metal surfaces is additionally verified or determined by a reference method. The content of salt deposits on the reference metal surfaces is preferably determined quantitatively by using the Bresle test and/or by difference weighing of the metal substrate used for referencing before and after contacting with a salt of known composition.

The determining of the area beneath one or more characteristic spectral lines (of a spectrum obtained by laser-induced plasma spectroscopy) in combination with the simultaneous ascertaining of the salt content on the same reference metal surface in each case makes it possible to assign a specific content of salt deposits or a specific content of the salt-forming chemical elements respectively present to the ascertained area beneath the respective characteristic spectral lines. By comparing the area of the spectral lines determined on the reference metal surfaces with the areas of the characteristic spectral lines (of the same species in each case) of the metal surface to be examined, it is then possible, by virtue of the linear correlation that exists between the size of the area beneath a characteristic spectral line and the content of salt deposits (or the content of the particular salt-forming chemical element being considered), to infer the quantitative amount of salt deposits (or the amount of respective salt-forming chemical element) on the metal surface to be examined.

Calibration measurements are conducted for the method of the invention at least on one reference metal surface having a known content of salt deposits. Preferably, for the method of the invention, calibration measurements are conducted on two or more reference metal surfaces with respectively different known contents of salt deposits. The measurement points thus obtained can then (according to the characteristic spectral line identified or used for quantitative detection) form a calibration line via interpolation, which can be utilized for comparison with the measurement results of the metal surface to be examined.

The calibration measurements for the method of the invention are preferably each conducted on reference metal surfaces having a respectively similar or identical chemical composition to the metal surface to be examined.

The expression “similar chemical composition” means that the main chemical element of reference metal surface and metal surface to be examined is the same, where the two metal surfaces may differ in any other proportions of chemical elements present in the metal surface. For the examination of steel surfaces, the calibration measurements should, for example, likewise be effected on steel surfaces as reference metal surfaces, although it is fundamentally immaterial for the calibration measurements which exact alloy additions are present in the steel surfaces used for the calibration measurements.

Nor is it necessary, for example, to conduct a separate calibration for each type of steel. Instead, it is sufficient, for example, to implement calibration measurements once on one particular type of (unalloyed) construction steels for examination of (unalloyed) construction steels in general, in which case these calibration measurements can also be used for quantitative detection of salt deposits on surfaces of various other types of (unalloyed) construction steel.

Preference is given to a method of the invention for quantitative detection of salt deposits (or of salt-forming chemical elements) on a metal surface by laser-induced plasma spectroscopy, comprising, as additional steps:
III. 1) identifying a spectral line characteristic of the metal surface to be examined from the spectrum obtained in step II) and determining the area beneath the spectral line;
III.2) forming an area ratio from the areas determined in steps III) and III.1);
wherein, in step IV), the content of salt deposits (or of salt-forming chemical elements) on the metal surface to be examined is quantified by comparing the area ratio obtained in step III.2) with area ratios obtained from the one or more calibration measurements.

The use of an area ratio (formed from the area beneath a characteristic spectral line for a chemical element present in the salt deposit and the area beneath a spectral line characteristic of the metal surface to be examined) here improves the accuracy and reproducibility of the quantification of the content of salt deposits or of salt-forming chemical elements on the metal surface to be examined.

Where an area ratio is used for quantification, the comparison of the area ratios formed is naturally made in step IV) with (calibration) area ratios that have each been formed from the areas of spectral lines of the same species. Accordingly, a particular option, especially when using an area ratio for the quantification, is that the calibration measurements are conducted on reference metal surfaces having respectively similar or identical chemical composition to the metal surface to be examined.

In the context of the present invention, characteristic spectral lines for the metal surface to be examined generally mean the spectral lines of all elements from which the metal surface is formed.

The characteristic spectral line used for the metal surface to be examined is preferably a spectral line of the main chemical element in the metal surface. For example, an iron spectral line is thus chosen in the case of the examination of steel surfaces, and an aluminum spectral line in the case of examination of an aluminum surface.

Further preferably, characteristic spectral lines chosen for the metal surface to be examined are a high-intensity, more preferably the highest-intensity, spectral line of an element of the metal surface. Lower-intensity spectral lines of elements of the metal surface to be examined are also alternatively suitable, provided that the intensity of the respective spectral line is sufficiently high that the area beneath it can be determined.

In choosing the characteristic spectral line for the metal surface to be examined, it is typically ensured that no spectral line of an element that could simultaneously be a constituent of a salt deposit to be detected is chosen.

Preference is given to a method of the invention for quantitative detection of salt deposits (or of salt-forming chemical elements) on a metal surface by laser-induced plasma spectroscopy, wherein steps I) to IV) are repeated at one or more further sites on the metal surface to be examined.

In other words, preference is thus given to a method of the invention in which the content of salt deposits or salt-forming chemical elements is ascertained at multiple sites on the metal surface to be examined. Measurement or verification of the salt content at multiple sites on the metal surface to be examined gives a more meaningful conclusion as to the presence of any salt contamination on the metal surface to be examined.

For the purposes of a further evaluation, the measurements obtained at multiple sites may, for example, be averaged (in order to obtain a statistically assured result) or accumulated. In addition, measurement at multiple sites can achieve scanning or mapping of the surface to be examined.

Further preferably, a multiple measurement is conducted at one and the same site on the metal surface to be examined. Such a multiple measurement at one site may be advantageous in order to verify whether, or to what extent, the individual measurements are subject to possible fluctuation.

Preference is given to a method of the invention for quantitative detection of salt deposits (or of salt-forming chemical elements) on a metal surface by laser-induced plasma spectroscopy, wherein steps III) to IV) are repeated for one or more further spectral lines that are characteristic of the salt deposits. With regard to the advantages of considering multiple spectral lines that are characteristic of the salt deposits, reference is made to the elucidations above.

Preference is likewise given to a method of the invention for quantitative detection of salt deposits (or of salt-forming chemical elements) on a metal surface by laser-induced plasma spectroscopy, wherein the metal surface to be examined is a steel surface, galvanized steel surface or aluminum surface, preferably a steel surface of a steel for steel construction or a galvanized steel surface. The aluminum surface to be examined is preferably a thermally sprayed aluminum surface.

The metal surface to be examined is more preferably a steel surface of a steel for steel construction, as specified in table 1 of standard DIN EN 10027-1:2017-01.

Preference is given to a method of the invention for quantitative detection of salt deposits (or of salt-forming chemical elements) on a metal surface by laser-induced plasma spectroscopy, wherein the spectral line which is characteristic of the salt deposits and is identified in step III) is a spectral line of an element selected from the group consisting of sodium, calcium, magnesium, potassium, chlorine, sulfur, nitrogen, phosphorus, carbon, bromine, iodine and oxygen, preferably a spectral line of an element selected from the group consisting of sodium, calcium, potassium and magnesium,
wherein the spectral line which is characteristic of the salt deposits and is identified in step III) is preferably selected from the group consisting of sodium spectral lines having a wavelength in the range from 300 to 600 nm, calcium spectral lines having a wavelength in the range from 200 to 600 nm, potassium spectral lines having a wavelength in the range from 400 to 800 nm and magnesium spectral lines having a wavelength in the range from 200 to 550 nm,
   wherein the spectral line which is characteristic of the salt deposits and is identified in step III) is more preferably selected from the group consisting of a sodium spectral line at a wavelength of 589.0 nm, a sodium spectral line at a wavelength of 589.6 nm, a calcium spectral line at a wavelength of 393 nm, a calcium spectral line at a wavelength of 396.8 nm, a potassium spectral line at a wavelength of 766.5 nm, a potassium spectral line at a wavelength of 769.9 nm, a magnesium spectral line at a wavelength of 279.6 nm and a magnesium spectral line at a wavelength of 280.3 nm,
      wherein the spectral line which is characteristic of the salt deposits and is identified in step III) is most preferably selected from the group consisting of a sodium spectral line at a wavelength of 589.0 nm and a sodium spectral line at a wavelength of 589.6 nm.

As already set out above, the use of high-intensity spectral lines is preferred. However, the method can likewise be conducted using less intense spectral lines, for example using sodium spectral lines at a wavelength of 330 nm.

It is also possible with the aid of the method of the invention to quantify carbon-containing salt deposits on a steel surface since a clean steel surface (i.e. one not having salt deposits) has less intense carbon spectral lines in the LIBS spectrum compared to a steel surface having carbon-containing salt deposits. Using calibration measurements, for example, on reference metal surfaces or reference steel surfaces with an equal carbon content to the steel surface to be examined, it is then possible to eliminate the intensity of the carbon spectral lines originating from the steel surface.

Preference is given to a method of the invention for quantitative detection of salt deposits (or of salt-forming chemical elements) on a metal surface by laser-induced plasma spectroscopy, wherein the salt deposits on the metal surface to be examined are salt deposits caused by seawater, deicing salt, industrial waste gases and/or emissions from the agricultural sector.

Preference is likewise given to a method of the invention for quantitative detection of salt deposits (or of salt-forming chemical elements) on a metal surface by laser-induced plasma spectroscopy, wherein the salt deposits on the metal surface to be examined or some of the salt deposits on the metal surface to be examined are salt deposits caused by jetting medium used in a jetting process (for example for cleaning of the metal surface to be examined prior to application of a coating).

Preference is given to a method of the invention for quantitative detection of salt deposits (or of salt-forming chemical elements) on a metal surface by laser-induced plasma spectroscopy, wherein the quantitative detection of salt deposits is conducted prior to coating of the metal surface to be examined. As already mentioned further up, verification of the salt content on a metal surface to be coated prior to the coating operation can reduce or entirely prevent later bubble formation (caused or promoted by salt deposits) in the coating to be applied.

Preference is given to a method of the invention for quantitative detection of salt deposits (or of salt-forming chemical elements) on a metal surface by laser-induced plasma spectroscopy, comprising, as an additional step:
V.a) repeating steps I) to IV) in the immediate proximity of those sites on the metal surface to be examined that have already been analyzed where a previously fixed limit for the content of salt deposits has been exceeded,
and/or
V.b) cleaning those sites on the metal surface to be examined where a previously fixed limit for the content of salt deposits is exceeded, wherein the previously fixed limit for the content of salt deposits is preferably 20 mg/m² of sodium chloride equivalents.

If the measurement result obtained by the method of the invention should show exceedance of a limit of salt deposits that are still acceptable on the metal surface to be examined, it is advisable to define the extent of salt contamination by further measurements and then to (re) clean the salt-contaminated site prior to coating of the metal surface.

Preferably, after the (re) cleaning, one or more further measurements should be conducted in order to check the outcome of the cleaning.

Preference is given to a method of the invention for quantitative detection of salt deposits (or of salt-forming chemical elements) on a metal surface by laser-induced plasma spectroscopy, wherein
the one or more reference metal surfaces are each obtained by
   contacting a cleaned metal surface with a salt-containing aerosol, preferably with a salt-containing mist, over a defined period of time,
   and optionally a drying step that takes place after the contacting with the salt-containing aerosol,
wherein the salt-containing aerosol comprises one or more salts (in solid or dissolved form) and the chemical composition of the one or more salts encompassed by the salt-containing aerosol is known in each case and, when there are two or more salts, the relative ratio of the amounts of salts to one another is known,
and
the content of salt deposits is referenced to each of the one or more reference metal surfaces via at least one measurement method other than laser-induced plasma spectroscopy, preferably via difference weighing before and after contacting with the salt-containing aerosol and/or via the Bresle method according to DIN EN ISO 8502-6:2020-08 and DIN EN ISO 8502-9:2020-12.

Contacting with a salt-containing aerosol has been found to be particularly advantageous in order to obtain reference metal surfaces having defined and homogeneous (uniform) contents of salt deposits.

By variation of, for example, the duration for the contacting and the concentration of salt in the aerosol, it is possible here to establish the ultimate concentration of salt deposits on the reference metal surfaces.

One advantage of contacting with a salt-containing aerosol is that this results in no contact of liquid (when a suspended dust is used, i.e. a mixture of finely divided particles in a gas, as aerosol) or in contact with only a small amount of liquid (when a salt-containing mist is used, i.e. finely divided liquid droplets of a salt-containing solution in a gas, as aerosol) with the metal surface to be contacted, hence ruling out or greatly minimizing risk of corrosion to the reference metal surface.

In principle, in the case of contact with a salt-containing mist, drying at room temperature (20° C.) and atmospheric pressure is sufficient. However, when a salt-containing mist is used, the drying step is preferably effected with supply of heat (for example via a hot plate or inductive heating) and/or by reduction in ambient pressure (for example by drying in a desiccator).

When a drying step takes place after contact, in the case of referencing of the contacted salt content via difference weighing, the second weighing is effected only after the end of the drying step.

In the case of referencing of the contacted salt content via the Bresle method (the Bresle test), the reference metal surface must be at least sufficiently large that, alongside the required space (i.e. the required measurement point) for the laser-induced plasma spectroscopy to be conducted, it is also possible to stick on at least one chamber patch (Bresle patch) for the Bresle test.

Preference is given to a method of the invention for quantitative detection of salt deposits (or of salt-forming chemical elements) on a metal surface by laser-induced plasma spectroscopy, wherein,
in step II) of spectroscopy analysis, an exposure time of a detector (gate width) in the range from 1 us to 100 us is chosen, preferably in the range from 1 us to 10 μs, more preferably of 10 μs,
and/or
an interval between a laser pulse and the measurement of a spectrum (gate delay) in the range from 0.5 us to 5 us is chosen, preferably in the range from 0.5 us to 2 μs, more preferably of 2 μs.

As already mentioned, a significant part of what is achieved by the invention is development of a methodology by which a metal surface can be contacted homogeneously, in a simple and efficient manner, with a defined content of salt deposits, which can then be used as reference metal surfaces for calibration measurements.

Also part of the invention, therefore, is a method of uniformly contacting a metal surface with salts or of producing a reference metal surface (as defined above and in the claims), comprising the following steps:
providing a metal substrate having a cleaned metal surface or cleaning the metal surface of a metal substrate,
providing an (aerosol) chamber or an (aerosol) vessel comprising an aerosol inlet and an aerosol outlet,
positioning the metal substrate having a cleaned metal surface in the chamber or the vessel and/or in the vicinity of the aerosol outlet,
contacting the metal surface with salts by introducing a salt-containing aerosol into the chamber or the vessel,
   wherein the salt-containing aerosol comprises one or more salts (in solid or dissolved form) and the chemical composition of the one or more salts encompassed by the salt-containing aerosol is known in each case, and, in the case of two or more salts, the ratio of the amounts of salts to one another is known.

This mode of production of reference metal surfaces (as already mentioned above) has the advantage that risk of corrosion of the reference metal surface is ruled out or greatly minimized. The use of an (aerosol) chamber or of an (aerosol) vessel serves the purpose of minimizing the effect of air movements of the ambient air of the contacting process. By variation of, for example, the duration for contacting and the concentration of salt in the aerosol, it is possible to adjust the ultimate concentration of salt deposits on the reference metal surfaces.

The wording of “positioning the metal substrate in the vicinity of the aerosol outlet” encompasses both positioning of the metal substrate in the vicinity of the aerosol outlet within the chamber or vessel and positioning of the metal substrate close to the aerosol outlet outside the chamber or vessel.

Preference is given to a method of simultaneously contacting a metal surface with salts or of producing a reference metal surface (as defined above and in the claims), wherein
the metal substrate with a cleaned metal surface is positioned in the vicinity of the aerosol outlet, preferably in the immediate proximity of the aerosol outlet outside the chamber or vessel (i.e. immediately beneath the aerosol outlet),
and/or
the chamber or the vessel additionally comprises a cover for indirect aerosol contacting.

For the creation of sample series, the metal substrates are preferably always positioned in the same place and at the same distance from the aerosol outlet.

For the purposes of the method of the invention for uniform contacting of a metal surface with salts or of production of a reference metal surface (as defined above and in the claims), the metal substrate may be positioned, for example, by laying or hanging.

In principle, it is alternatively also possible to obtain reference metal surfaces for the calibration measurements in the method of the invention for quantitative detection of salt deposits on a metal surface by other ways known to the person skilled in the art; for example by immersing a metal substrate into a salt-containing solution or by painting a metal substrate with a salt-containing solution.

In addition, it is possible to achieve uniform contacting of a metal surface with salts or the production of a reference metal surface (as defined above and in the claims) by a method comprising the following steps:
providing a metal substrate having a cleaned metal surface or cleaning the metal surface of a metal substrate,
applying a salt deposit of known composition with the aid of an inkjet printer.

Also part of the invention is an apparatus for quantitative detection of salt deposits (or of salt-forming chemical elements) on a metal surface by laser-induced plasma spectroscopy by a method of the invention (as defined above and in the claims), comprising
a laser for creation of a (local) plasma on the metal surface to be examined;
a spectrometer set up to detect a spectrum of a plasma emission radiation, and
an evaluation unit,
   wherein the evaluation unit comprises one or more stored calibration data,
preferably one or more areas formed in one or more spectra from calibration measurements and using step III) of the method of the invention for quantitative detection of salt deposits (or of salt-forming chemical elements) on a metal surface by laser-induced plasma spectroscopy,
   more preferably one or more area ratios formed in one or more spectra from calibration measurements and using steps III), III.1) and III.2) of the preferably method of the invention for quantitative detection of salt deposits (or of salt-forming chemical elements) on a metal surface by laser-induced plasma spectroscopy,
      and the evaluation unit is set up to quantify salt deposits on a metal surface by comparison of a detected spectrum of a plasma emission radiation with the one or more stored calibration data.

An apparatus of the invention preferably additionally comprises a focusing unit for focusing a laser beam on the metal surface.

Preference is also given to an apparatus of the invention, wherein the laser
is set up to scan the metal surface
and/or
is an Nd:YAG laser
and/or
emits radiation with a wavelength of 266 nm, 532 nm or 1064 nm
and/or
is a pulsed laser, preferably with a pulse energy in the range from 10 mJ to 250 mJ and/or with a pulse length of 1 ps to 10 ns, more preferably with a pulse energy of 45 mJ and a pulse length of 8 ns.

What is meant by “to scan the metal surface” is that the laser is set up such that it can be moved across the metal surface. Additionally or alternatively, the apparatus may also be set up to permit movement (a change in the position) of the metal surface to be examined.

Preference is likewise given to an apparatus of the invention wherein
the spectrometer is an Echelle spectrometer, preferably having a CCD sensor as detector,
and/or
the apparatus is transportable
and/or
the apparatus is configured in the form of a handheld device or in the form of an automatic measurement device, preferably a robot-assisted automatic measurement device.

In a further preferred embodiment, the inventive may additionally be a stationary apparatus (tied to a fixed location). Configuration as a stationary apparatus is preferred particularly for laboratory applications.

Likewise part of the invention is the use of laser-induced plasma spectroscopy for quantitative detection of salt deposits on a metal surface.

Preference is given here to the use of laser-induced plasma spectroscopy for quantitative detection of salt deposits on metal surfaces to be coated, more preferably on metal surfaces to be coated for steel construction, steel hydraulics construction, shipbuilding, offshore technology, energy and plant technology, the automotive sector and aerospace.

Also part of the invention is the use of an apparatus of the invention (as defined above and in the claims) for performance of a method of the invention for quantitative detection of salt deposits (or of salt-forming chemical elements) on a metal surface by laser-induced plasma spectroscopy (as defined above and in the claims).

The invention is elucidated in detail below by examples and the appended figures. The examples cited below are intended to describe and explain the invention in detail without restricting its scope.

The figures show:

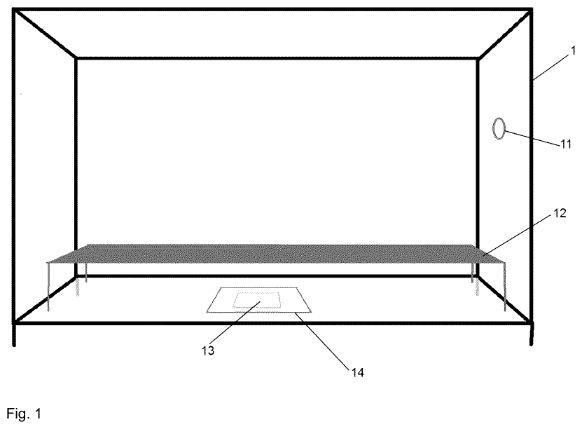

FIG. 1: Schematic diagram of an aerosol chamber for uniform contacting of a metal surface with salts or for production of a reference metal surface.

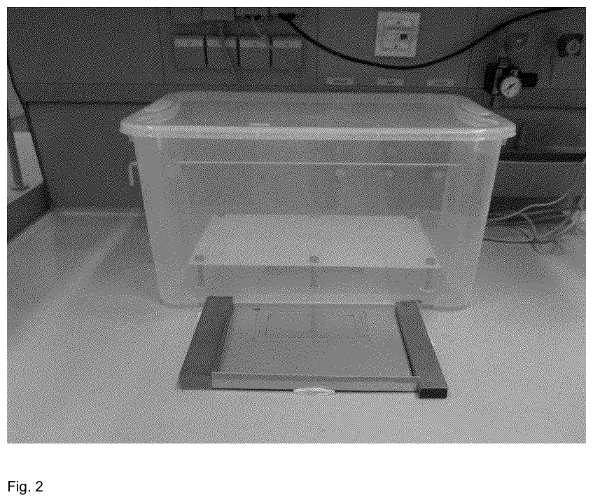

FIG. 2: Image (side view) of an aerosol chamber for uniform contacting of a metal surface with salts or for production of a reference metal surface. A tablet for transport is apparent in front of the aerosol chamber, and the positioning of the metal substrates beneath the aerosol chamber.

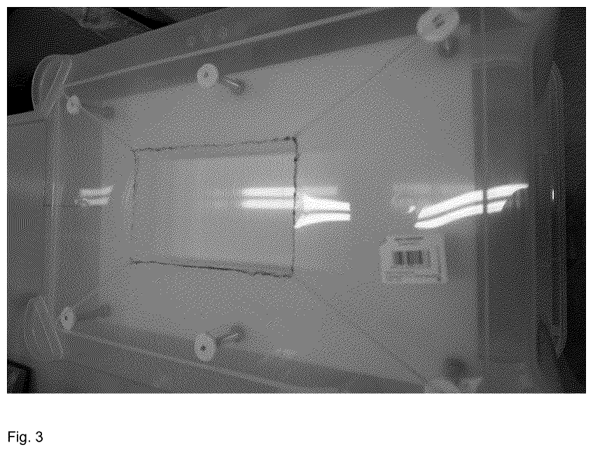

FIG. 3: Image of the aerosol outlet of an aerosol chamber for uniform contacting of a metal surface with salts or for production of a reference metal surface.

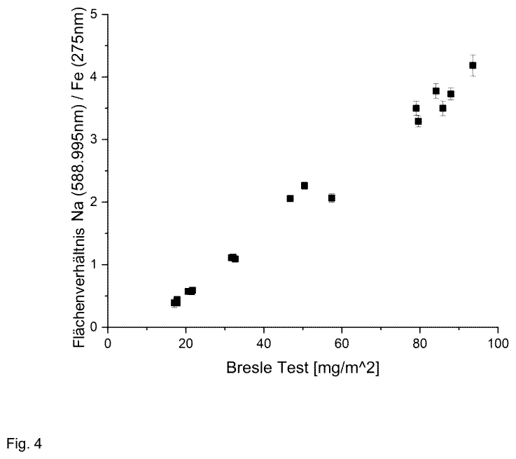

FIG. 4: Juxtaposition of the area ratio obtained by laser-induced plasma spectroscopy from the area beneath the sodium spectral line at 588.995 nm and the area beneath the iron spectral line at 275 nm with the salt content quantified by the Bresle test (reported in NaCl equivalents) on reference metal surfaces of steel specimens having different contents of salt deposits on the surface.

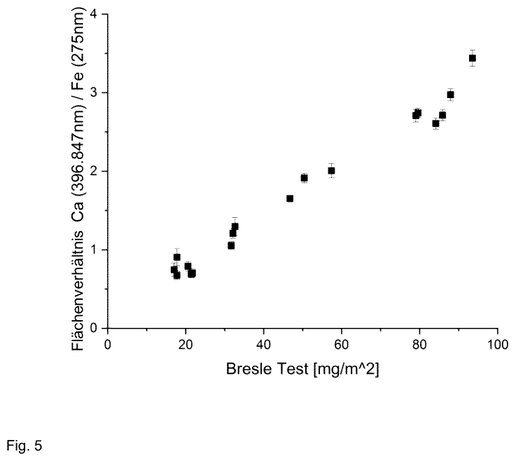

FIG. 5: Juxtaposition of the area ratio obtained by laser-induced plasma spectroscopy from the area beneath the calcium spectral line at 396.847 nm and the area beneath the iron spectral line at 275 nm with the salt content quantified by the Bresle test (reported in NaCl equivalents) on reference metal surfaces of steel specimens having different contents of salt deposits on the surface.

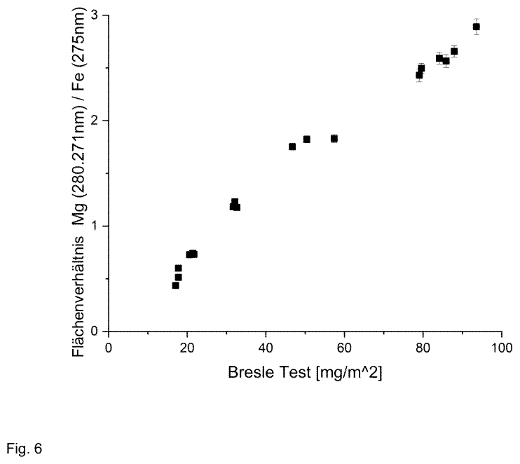

FIG. 6: Juxtaposition of the area ratio obtained by laser-induced plasma spectroscopy from the area beneath the magnesium spectral line at 280.271 nm and the area beneath the iron spectral line at 275 nm with the salt content quantified by the Bresle test (reported in NaCl equivalents) on reference metal surfaces of steel specimens having different contents of salt deposits on the surface.

### PRODUCTION OF REFERENCE METAL SURFACES HAVING A KNOWN CONTENT OF SALT DEPOSITS AND CORRELATION BETWEEN THE SALT CONTENT ASCERTAINED BY THE BRESLE TEST AND THE AREA RATIOS OF CHARACTERISTIC SPECTRAL LINES OBTAINED BY LASER-INDUCED PLASMA SPECTROSCOPY

A metal substrate in the form of a steel specimen was cleaned and positioned beneath an aerosol chamber in the immediate proximity of the aerosol outlet of the aerosol chamber located therein. The aerosol chamber used is shown schematically in FIG. 1 and depicted in FIGS. 2 and 3.

On completion of positioning, the steel specimen was contacted with a salt-containing mist over a defined period. For this purpose, the salt-containing mist was introduced into the aerosol chamber via the aerosol inlet and, after exiting from the aerosol outlet, arrived at the steel specimen positioned beneath. In the aerosol chamber, above the aerosol outlet, there was a cover for indirect aerosol contacting, which ensured uniform distribution of the salt-containing mist over the entire width of the aerosol outlet.

For the production of the salt-containing mist, synthetic seawater was used as the liquid component. The synthetic seawater was obtained here by producing two individual solutions from the constituents specified in table 1, followed by mixing of the two solutions 1 and 2.

TABLE 1
Compositions of solutions 1 and 2 for production of
synthetic seawater (in accordance with DIN 50905).

| Solution 1 | | Solution 2 | |
|---|---|---|---|
| 1770 | mL of distilled water (H₂O) | 200 | mL of distilled water (H₂O) |
| 56 | g of sodium chloride (NaCl) | 0.40 | g of sodium hydrogencarbonate |
| | | | (NaHCO₃) |
| 10 | g of magnesium chloride (MgCl₂) | 14 | g of magnesium sulfate (MgSO₄) |
| 4.8 | g of calcium chloride (CaCl₂) | | |

To create a salt-containing mist from the synthetic seawater, the synthetic seawater was introduced into an inhalation system and nebulized therewith. For this purpose, an inhalation system which is typically used for medical purposes was used. The inhalation system used (Compact 2) from Pari consisted of a compressor, an atomizer and an outlet.

After the predefined period for mist treatment of the steel specimen had ended, it was removed from the aerosol outlet and, on completion of drying, the salt content on the surface of the steel specimen contacted with salt was quantified by employing the Bresle test according to DIN EN ISO 8502-6:2020-08 with performance of a conductivity measurement according to DIN EN ISO 8502-9:2020-12. In addition, at other sites on the surface of the steel specimen that had been contacted with salt, measurements were effected by laser-induced plasma spectroscopy.

The salt content ascertained with the aid of the Bresle test was converted to NaCl equivalents, and compared with the area ratios obtained by laser-induced plasma spectroscopy, in each case formed from the area beneath a characteristic spectral line for a chemical element present in the salt deposit and the area beneath a spectral line characteristic of the steel surface.

The procedure elucidated above was repeated for further steel specimens, in each case varying the period of contacting with a salt-containing mist in order to obtain a set of reference metal surfaces having different contents of salt deposits on the metal surface. The steel specimens to be contacted were each positioned beneath the aerosol chamber in the same place and at the same distance from the aerosol outlet.

FIGS. 4, 5 and 6 show the juxtaposition by way of illustration, in graph form, of the salt contents of the reference metal surfaces produced, as obtained by the Bresle test, with selected area ratios (obtained by laser-induced plasma spectroscopy). It can be seen from each of FIGS. 4, 5 and 6 that a linear correlation exists between the salt content on the reference metal surfaces and the size of the area ratio obtained by laser-induced plasma spectroscopy.

The calibration measurements thus obtained were subsequently utilized to run quantitative detection of salt deposits on the surface of the steel substrate by comparison of the calibration data with LIBS measurements on a surface of a steel substrate (that has been exposed to seawater).

### LIST OF REFERENCE NUMERALS

1 aerosol chamber for uniform contacting of a metal surface with salts or for production of a reference metal surface
11 aerosol inlet
12 cover for indirect aerosol contacting
13 metal substrate with cleaned metal surface
14 aerosol outlet

1. A method of quantitatively detecting salt deposits on a metal surface by laser-induced plasma spectroscopy, comprising the following steps:
I) focusing a laser beam onto a point on the metal surface to be examined, so as to form a local plasma;
II) spectroscopically analyzing the radiation emitted by the local plasma on cooling, so as to obtain a spectrum for the radiation emitted by the local plasma;
III) identifying one characteristic spectral line for at least one chemical element present in the salt deposit from the spectrum obtained in step II) and determining the area beneath the spectral line;
IV) quantifying the content of salt deposits on the metal surface to be examined by comparing the area determined in step III) with areas obtained from calibration measurements,
wherein the calibration measurements are each performed on one or more reference metal surfaces with respectively known contents of salt deposits.

2. The method as claimed in claim 1, comprising, as additional steps:
III. 1) identifying a spectral line characteristic of the metal surface to be examined from the spectrum obtained in step II) and determining the area beneath the spectral line;
III.2) forming an area ratio from the areas determined in steps III) and III.1);
wherein, in step IV), the content of salt deposits on the metal surface to be examined is quantified by comparing the area ratio obtained in step III.2) with area ratios obtained from the one or more calibration measurements.

3. The method as claimed in claim 1, wherein steps I) to IV) are repeated at one or more further sites on the metal surface to be examined.

4. The method as claimed in claim 1, wherein steps III) to IV) are repeated for one or more further spectral lines that are characteristic of the salt deposits.

5. The method as claimed in claim 1, wherein the metal surface to be examined is a selected from the group consisting of a steel surface, a galvanized steel surface, an aluminum surface, and a steel surface of a steel for steel construction.

6. The method as claimed in claim 1, wherein the spectral line which is characteristic of the salt deposits and is identified in step III) is a spectral line of an element selected from the group consisting of sodium, calcium, magnesium, potassium, chlorine, sulfur, nitrogen, phosphorus, carbon, bromine, iodine and oxygen.

7. The method as claimed in claim 2, wherein the spectral line which is characteristic of the metal surface to be examined and is identified in step III.1) is a spectral line of an element selected from the group consisting of iron, zinc and aluminum.

8. The method as claimed in claim 1, wherein the salt deposits on the metal surface to be examined are salt deposits caused by at least one of seawater, deicing salt, industrial waste gases, emissions from the agricultural sector, and a combination of at least two of the aforementioned.

9. The method as claimed in claim 1, wherein the quantitative detection of salt deposits is conducted prior to coating of the metal surface to be examined.

10. The method as claimed in claim 1, comprising, as an additional step:
repeating steps I) to IV) in the immediate proximity of those sites on the metal surface to be examined that have already been analyzed where a previously fixed limit for the content of salt deposits has been exceeded.

11. The method as claimed in claim 1,
wherein
the one or more reference metal surfaces are each obtained by
contacting a cleaned metal surface with a salt-containing aerosol, preferably with a salt-containing mist, over a defined period of time,
wherein the salt-containing aerosol comprises one or more salts and the chemical composition of the one or more salts encompassed by the salt-containing aerosol is known in each case and, when there are two or more salts, the relative ratio of the amounts of salts to one another is known,
and
the content of salt deposits is referenced to each of the one or more reference metal surfaces via at least one measurement method other than laser-induced plasma spectroscopy.

12. The method as claimed in claim 1, wherein,
in step II) of spectroscopy analysis, an exposure time of a detector is chosen in the range from 1 μs to 100.

13. A method of uniformly contacting a metal surface with salts or of producing a reference metal surface as defined in claim 1, comprising the following steps:
providing a metal substrate having a cleaned metal surface (13) or cleaning the metal surface of a metal substrate,
providing a chamber (1) or a vessel comprising an aerosol inlet (11) and an aerosol outlet (14),
positioning the metal substrate having a cleaned metal surface (13) in the chamber (1) or the vessel and/or in the vicinity of the aerosol outlet (14),
contacting the metal surface with salts by introducing a salt-containing aerosol into the chamber (1) or the vessel,
wherein the salt-containing aerosol comprises one or more salts and the chemical composition of the one or more salts encompassed by the salt-containing aerosol is known in each case, and, in the case of two or more salts, the ratio of the amounts of salts to one another is known.

14. The method as claimed in claim 13, wherein
the metal substrate with a cleaned metal surface (13) is positioned in the vicinity of the aerosol outlet (14).

15. An apparatus for quantitative detection of salt deposits on a metal surface by laser-induced plasma spectroscopy by a method as defined in claim 1, comprising
a laser for creation of a plasma on the metal surface to be examined;
a spectrometer set up to detect a spectrum of a plasma emission radiation, and
an evaluation unit,
wherein the evaluation unit comprises one or more stored calibration data,
and the evaluation unit is set up to quantify salt deposits on a metal surface by comparison of a detected spectrum of a plasma emission radiation with the one or more stored calibration data.

16. The apparatus as claimed in claim 15, additionally comprising a focusing unit for focusing a laser beam on the metal surface.

17. The apparatus as claimed in claim 15, wherein the laser is set up to scan the metal surface.

18. The apparatus as claimed in claim 15, wherein the spectrometer is an Echelle spectrometer.

19. A method for quantitative detection of salt deposits on a metal surface comprising analyzing the metal surface with laser-induced plasma spectroscopy.

20. The method as claimed in claim 1, wherein the method is performed with an apparatus comprising:
a laser for creation of the local plasma on the metal surface to be examined;
a spectrometer set up to detect a spectrum of a plasma emission radiation, and
an evaluation unit,
wherein the evaluation unit comprises one or more stored calibration data, and the evaluation unit is set up to quantify salt deposits on a metal surface by comparison of a detected spectrum of a plasma emission radiation with the one or more stored calibration data.

21. The method as claimed in claim 6, wherein the spectral line which is characteristic of the salt deposits and is identified in step III) is selected from the group consisting of sodium spectral lines having wavelengths in the range from 300 to 600 nm, calcium spectral lines having wavelengths in the range from 200 to 600 nm, magnesium spectral lines having wavelengths in the range from 200 to 550 nm, and potassium spectral line having a wavelength in the range from 400 to 800 nm.

22. The method as claimed in claim 7, wherein the spectral line which is characteristic of the metal surface to be examined and is identified in step III.1) is selected from the group consisting of iron spectral lines having wavelengths in the range from 200 to 600 nm, zinc spectral lines having wavelengths in the range from 200 to 650 nm and aluminum spectral lines having wavelengths in the range from 200 to 400 nm.

23. The method as claimed in claim 1, comprising, as an additional step of cleaning those sites on the metal surface to be examined where a previously fixed limit for the content of salt deposits is exceeded.

24. The method as claimed in claim 23, wherein the previously fixed limit for the content of salt deposits is 20 mg/m² of sodium chloride equivalents.

25. The method as claimed in claim 11, wherein the one or more reference metals are each obtained by conducting a drying step after the contacting with the salt-containing aerosol.

26. The method as claimed in claim 11, wherein the content of salt deposits is referenced to each of the one or more reference metal surfaces via the at least one measurement method other than laser-induced plasma spectroscopy selected from the group consisting of a difference weighing before and after contacting with the salt-containing aerosol, a Bresle method according to DIN EN ISO 8502-6:2020-08 and DIN EN ISO 8502-9:2020-12, and a combination of the aforementioned methods.

27. The method as claimed in claim 1, an interval between a laser pulse and the measurement of a spectrum in the range from 0.5 us to 5 us is chosen, preferably in the range from 0.5 us to 2 μs, more preferably of 2 μs.

28. The method as claimed in claim 13, wherein the chamber (1) or the vessel additionally comprises a cover for indirect aerosol contacting (12).

29. The apparatus as claimed in claim 15, wherein the evaluation unit comprises one or more areas formed in one or more spectra from calibration measurements and using step III).

30. The apparatus as claimed in claim 15, wherein the evaluation unit comprises one or more area ratios formed in one or more spectra from calibration measurements and using steps:
III) identifying the at least one characteristic spectral line for the at least one chemical element present in the salt deposit from the spectrum obtained in step II) and determining the area beneath the spectral line;
III. 1) identifying the spectral line characteristic of the metal surface to be examined from the spectrum obtained in step II) and determining the area beneath the spectral line; and
III.2) forming the area ratio from the areas determined in steps III) and III.1).

31. The apparatus as claimed in claim 15, wherein the laser is selected from the group consisting of an Nd:YAG laser; a laser configured to emit radiation with a wavelength selected from the group consisting of 266 nm, 532 nm and 1064 nm; a pulsed laser; a pulsed laser having a pulse energy in the range from 10 mJ to 250 mJ; and, a pulsed laser having a pulse length in a range of 1 ps to 10 ns.

32. The apparatus as claimed in 18, wherein the Echelle spectrometer has a CCD sensor as detector.

33. The apparatus as claimed in 15, wherein the apparatus is selected from the group consisting of a transportable apparatus, the apparatus configured having a form of a handheld device, the apparatus configured having a form of an automatic measurement device, and the apparatus configured having a form of a robot-assisted automatic measurement device.
